(19) United States
(12) Patent Application Publication
(10) Pub. No.: US 2020/0214978 A1
(43) Pub. Date: Jul. 9, 2020

(54) Functional chewing gum comprising phytonutrients and adaptogenic herbs

(71) Applicant: BrilliantIQ AB, Tyresö (SE)

(72) Inventors: Roger Peterson, Stockholm (SE); Robin Peterson, Tyresö (SE); Ulf Gresté, Huddinge (SE)

(21) Appl. No.: 16/641,707

(22) PCT Filed: Aug. 27, 2018
(86) PCT No.: PCT/EP2018/073039

§ 371 Date: Feb. 25, 2020

(30) Foreign Application Priority Data
Aug. 25, 2017 (FI) 20175763

(51) Int. Cl. A61K 9/68; A61K 35/02; A61K 36/81; A61K 31/683; A61K 31/4412; A61K 36/68; A61K 36/258; A61K 31/4415; A61K 31/375; A61K 36/074; A61K 33/30; A61K 33/18; A61K 33/06; A61K 31/51; A61K 31/525; A61K 31/714; A61K 36/49; A61K 31/4375

(52) U.S. Cl. CPC A61K 9/0058; A61K 31/4375; A61K 36/81; A61K 31/683; A61K 31/4412; A61K 36/68; A61K 36/258; A61K 31/4415; A61K 31/375; A61K 36/074; A61K 33/30; A61K 33/18; A61K 33/06; A61K 31/51; A61K 31/525; A61K 31/714; A61K 36/49; A61K 35/02

(57) ABSTRACT

The present invention is directed to a functional chewing gum comprising a gum base and active ingredients boosting in mental clarity and concentration, wherein said active ingredients are selected among Shilajit, Ashwagandha, coenzyme Q10, L-Tirosine, Rhodiola, Bacopa monnieri, L-Alpha glycerylphosphorylcholine (alpha-GPC), Guayusa, Phosphatidylserine (preferably GMO free), and piperine (i.e. blackpepper extract). The present invention is also directed to a functional chewing gum comprising a gum base and active ingredients providing alleviation of fatigue and enhancement of energy, wherein said active ingredients are selected among Shilajit, coenzyme Q10, citrulline malate, Siberian Ginseng, L-Tyrosine, Ganoderma lucidum (Reishi), Hericium erinaceus (Lion's mane), Coconut Oil powder, Shilajit, Guayusa, Resveratrol and piperine (blackpepper extract).

### FIELD OF THE INVENTION

The present invention relates to the field of nutraceuticals and particularly to functional chewing gums comprising phytonutrients and adaptogenic herbs. The chewing gum of the present invention comprises a gum base and a balanced mix of phytonutrients and adaptogenic herbs, which in a synergistic manner provides a boost in mental clarity and concentration and/or alleviation of fatigue and enhancement of energy and/or function as circadian rhythm modulators to help counteract neurotransmitter imbalance and thus improve mood, mental clarity, and physical stamina.

### BACKGROUND OF THE INVENTION

Circadian rhythm disruption has been shown to have negative consequences in numerous biological systems in healthy subjects, in particular; immune, inflammatory and oxidative stress systems. Multiple mechanisms have been proposed to explain the correlation between sleep and oxidative stress, including sleep-related changes in transcriptional responses of genes involved in oxidative stress in peripheral tissues. However, not many medical treatments for circadian rhythm disorder has been disclosed.

There are several circadian transcription factors that control positive and negative feedback loops in the cells to determine functions of key organs and systems, including heart, lungs, immune system, and metabolism, as well as the intracellular processes, such as cellular respiration and DNA repair. This internal clock system regulates roughly one-third of the body's entire gene activity! The genetic code regulating these systems affects the body's timing for peak performance, such as whether you are a morning person or a night owl.

The central regulator of your circadian rhythm is the suprachiasmatic nucleus, SCN, which is found in the hypothalamus in the brain. It does this largely through controlling the secretion of melatonin, which is the main circadian hormone. Disruptions in the circadian rhythm also reduce life expectancy. Furthermore, as an individual ages, one becomes more sensitive to disruptions in the circadian rhythms—and less sensitive to synchronization techniques.

A recent study showed that patients with depression have an increased number of melatonin receptors in the SCN. An increase in melatonin receptors may be a compensatory mechanism for the reduced melatonin levels in patients with mood disorders, and these receptors have been recently suggested as targets of novel antidepressant agents.

Besides its role in circadian rhythm control, melatonin has antioxidant properties, acting as an electron donor in scavenging free radicals to protect against oxidative damage to lipids, proteins and DNA. Oxidative damage occurs when there is a disturbance in the oxidant—antioxidant balance, as a result of an overproduction of reactive oxygen species (ROS) and/or insufficient antioxidant defence. Melatonin is more effective at neutralising ROS than other intracellular antioxidants such as glutathione (GSH) and also stimulates antioxidant enzyme activity.

Shilajit is an important, known component of the ayurvedic medicine given its characteristics as a rasayana. In this context, health benefits such as an increase in longevity, rejuvenating, and arresting aging roles have been attributed to it. This phytocomplex known as shilajit is mainly composed of humic substances. One of them, fulvic acid, is known by its properties such as antioxidant, anti-inflammatory, and memory enhancer. Novel investigations indicate that fulvic acid is an antiaggregation factor of tau protein in vitro, which projects fulvic acid as a potential anti-Alzheimer's disease molecule. Shilajit is an ancient herbal substance containing over 85 minerals and trace elements the human body needs to function optimally and fulvic acid act as a catalyst for vitamin absorption for these minerals at a cellular level.

Ashwagandha and Rhodiola (Rhodiola rosea) are herbs that are used for stress management and to address other health ailments. While they share some similarities, each herb has unique properties. Ashwagandha, has been used to enhance energy, memory and endurance, as well as to alleviate stress, weakness, insomnia, arthritis, asthma, depressed immunity, sexual difficulties and infertility.

Ashwagandha contains the active compounds with anolides, which account for its therapeutic activity, especially pertaining to inflammation, immunity, memory and stress.

Rhodiola rosea, it's used to rejuvenate the nervous system, enhance work performance, combat fatigue, reduce stress, decrease depression and enhance fertility. The UMHS attributes rhodiola's medicinal properties to its phenylpropanoids, phenylethanol derivatives, flavonoids, monoterpenes, triterpenes and phenolic acids.

U.S. Pat. No. 8,636,985 discloses a nutritional gum comprising Ashwaganda herb and L-theanine.

The present invention provides a chewing gum with a balanced mix of phytonutrients and adaptogenic herbs. The present invention also provides a chewing gum providing relief of fatigue and additional energy.

### DETAILED DESCRIPTION OF THE INVENTION

As used herein, by the term “chewing gum” is meant any chewing gum such as slab or stick gums, center-filled chewing gum or compressed chewing gum.

By the term “gum base” is meant the mainly water-insoluble and hydrophobic gum base ingredients that are mixed together, typically before the bulk portion of the chewing gum is added. The “gum base” may contain gum base polymers and plasticizers, waxes, emulsifiers, fats and/or fillers. The gum base may thus designate the typical water-insoluble chewing gum components, which may be manufactured in a first step and subsequently mixed with the phytonutrients used in the invention in a second step.

Phytonutrients, adaptogenic herbs and other substances suitable for the present functional chewing gum are preferably the following:

Shilajit, an ancient herbal substance containing over 85 minerals and trace elements the human body needs to function optimally and fulvic acid which act as a catalyst for vitamin absorption for these minerals at a cellular level.

The active constituents of shilajit contain dibenzo-alpha-pyrones and related metabolites, small peptides (constituting non-protein amino acids), some lipids, and carrier molecules (fulvic acids).

Furthermore, dibenzo-a-pyrones have been hypothesized to participate in the electron transport inside the mitochondria, thus facilitating production of more ATP, leading to increased energy. Thus, shilajit is found to increase energy, among other beneficial qualities. Other molecules present in shilajit preparations are eldagic acid, some fatty acids, resins, latex, gums, albumins, triterpenes, sterols, aromatic carboxylic acids, 3,4-benzocoumarins, amino acids, polyphenols, and phenolic lipids.

Shilajit stimulates the growth process of skin cells at a molecular level. It improves the blood's ability to carry oxygen and iron. These properties of shilajit give people a healthy appearance and youthful glow. Likewise, Shilajit's minerals help to maintain the body's optimum energy metabolism rate which naturally slows down with age. This is achieved partly through balancing electrolyte levels, and by burning off excess fat. Ayurveda practitioners tout its ability to balance hormone levels that change and cause stress as we age.

Shilajit finds extensive use in Ayurveda, for diverse clinical conditions. For centuries people living in the isolated villages in Himalaya and adjoining regions have used Shilajit alone, or in combination with, other plant remedies to prevent and combat problems with diabetes (Tiwari, V. P., et al., “An interpretation of Ayurvedica findings on Shilajit,” J. Res. Indigenous Med. (1973) 8:57). Moreover being an antioxidant it will prevent damage to the pancreatic islet cell induced by the cytotoxic oxygen radicals (Bhattacharya S. K., “Shilajit attenuates streptozotocin induced diabetes mellitus and decrease in pancreatic islet superoxide dismutase activity in rats,” Phytother. Res. (1995) 9:41-4; Bhattacharya S. K., “Effects of Shilajit on biogenic free radicals,” Phytother. Res. (1995) 9:56-9; and Ghosal, S., et al., “Interaction of Shilajit with biogenic free radicals,” Indian J. Chem. (1995) 34B:596-602). It has been proposed that the derangement of glucose, fat and protein metabolism during diabetes, results into the development of hyperlipidemia. In one study, Shilajit produced significant beneficial effects in lipid profile in rats (Trivedi N. A., et al., “Effect of Shilajit on blood glucose and lipid profile in alloxan-induced diabetic rats,” Indian J. Pharmacol. (2004) 36(6):373-376).

As discussed, shilajit has been used to treat various ailments. It is also recommended as a performance enhancer. Fulvic acids (FAs) are reported to elicit many important roles in biological systems of plants, in animals as well as humans, including: improvement of bioavailability of minerals and nutrients, serve as electrolytes, detoxification of toxic substances including heavy metals, perform as antioxidants, and improvement of immune function.

The preferred amount of shilajit in the present gum with 400-2500 mg of Gum base is 50-400 mg.

Ashwagandha (Withania somnifera, Physalis somnifera), also known as winter cherry or Indian ginseng is both a tonic and a sedative due to its adaptogenic properties. Withania refers to the plants primary extract and somnifera literally means “sleep-inducing”. According to Sushruta, rasayanas are the substances that slow the ageing process, increase longevity and increase strength, both mental and physical. The documented potential benefits of the use of ashwagandha is impressive. Published studies and detailed benefits such as stabilizing, good for insomnia, erratic digestion, constipation and anxiety. It is calming while strengthening, giving energy while also settling the nervous system.

Ashwagandha enhances the function of the brain and nervous system and improves the memory. It improves the function of the reproductive system promoting a healthy sexual and reproductive balance. Being a powerful adaptogen, it enhances the body's resilience to stress. Ashwagandha improves the body's defense against disease by improving the cell-mediated immunity. It also possesses potent antioxidant properties that help protect against cellular damage caused by free radicals. Adaptogens are herbs that help in combating stress. Stress is a mental or emotional strain which, if regularly induced over time can lead to significant imbalance or disease. Adaptogens also can improve the body's performance and endurance.

The preferred amount of ashwagandha in the present gum with 400-2500 mg of Gum base is 25-500 mg.

Coenzyme Q10 (CoQ10), also known as ubiquinone or ubidecarenone, is a powerful, endogenously produced, intracellularly existing lipophilic antioxidant. It combats reactive oxygen species (ROS) known to be responsible for a variety of human pathological conditions. Its target site is the inner mitochondrial membrane (IMM) of each cell. Coenzyme Q10 (CoQ10), also identified as ubiquinone or ubidecarenone, is a natural fat-soluble, vitamin-like, ubiquitously existing benzoquinone derivative. It acts primarily as an antioxidant, a membrane stabilizer and a cofactor in the production of adenosine triphosphate (ATP) in the process well-known as oxidative phosphorylation. With such essential role, it steers cellular machinery and synthesis. Targeting cellular energy production in neurological disorders.

The preferred amount coenzyme Q10 in the present gum with 400-2500 mg of Gum base is 15-400 mg.

Phosphatidylserine (PS), an amino acid derivative that is highly prevalent in the neural tissue of humans. This fat-soluble phospholipid is found in every single cell membrane of your body, with high concentrations of it in the brain. Phosphatidylserine is especially important for cellular function in the brain. Phosphatidylserine is not a neurotransmitter but a special form of phospholipid which supports many neurotransmitters improving cognitive function, clearing depression, and enhancing memory and mood, phosphatidylserine (PS) blunts cortisol and helps us deal with stress.

Phosphatidylserine has a unique structure that makes it both hydrophilic, meaning it's attracted to water and hydrophobic, or repelled by water. With this structure, phospholipids are able to arrange themselves into a phospholipid bilayer—two parallel layers that form a major component of the human cell membranes. In brain cell membranes, the phospholipid bilayer acts as a “gatekeeper,” regulating the entry beneficial substances, basically nutrients, water and oxygen, while eliminating metabolic waste. Phosphatidylserine is required in the synthesis of Acetylcholine.

The most renowned benefit of phosphatidylserine is its potential for improving brain function. This nutrient is essential for keeping brain cells healthy and optimally functioning. It's also important for helping neurons conduct nerve impulses, supports your brain's ability to repair existing brain cells and produce new ones, and increases fluidity of brain cells, so they effectively respond to stressors. Due to these effects on the brain, PS is believed to produce the following brain health benefits. Boosts memory and cognitive function. A study found that supplementing with phosphatidylserine helped increase the speed of calculations done in short-term memory by 20 percent in a group of healthy adults.

In addition to glucose mediation, phosphatidylserine orchestrates balanced cortisol levels. The adrenal glands produce cortisol in a circadian rhythm: this hormone should peak in the morning and then gradually decrease until evening. When our cortisol levels fall out of this pattern, the body loses equilibrium. Chronic stress levels cause the adrenal glands to pump out mega-doses of cortisol, which then desensitize two parts of the brain called the hypothalamus and the hippocampus. Phosphatidylserine rebalances high cortisol not by blocking the production, but by re-sensitizing the hypothalamus and hippocampus to this hormone.

Phosphatidylserine has also shown potential for boosting athletic performance. For example, it may prolong the time it takes for a person who is exercising to become tired. It also reduces the amount of negative hormonal response to exercise, as shown by several studies. It also aids recovery from physical injury and may help a person recover from reduced blood flow.

Studies show Phosphatidylserine's potential for Alzheimer's disease, a condition that affects more than 5 million Americans today, is now the sixth leading cause of death in the country today. Alzheimer's disease prompts the brain to use glucose less efficiently, which is something that phosphatidylserine may also provide benefits for—it actually helps improve glucose metabolism in brains affected by this condition.

Phospholipids—with egg and soy being the main food sources. It is necessary that pharmaceutical agents be able to traverse cell membranes in order to facilitate their absorption, distribution, and elimination from the body. Due to their amphipathic nature, phospholipid systems are compatible with cell membranes. Lipid emulsion systems have appealing properties as drug carriers, due to their biodegradability, biocompatibility, stability, and ease of production.

L-Alpha glycerolphosphorylcholine (Alpha GPC). Alpha GPC is a natural choline compound found in the brain. It is also parasympathomimetic acetylcholine precursor which may have potential for the treatment of Alzheimer's disease and other dementias. Alpha-GPC rapidly delivers choline to the brain across the blood—brain barrier and is a biosynthetic precursor of acetylcholine.

Oral administration of alpha-GPC induced a significant decrease in delta waves (usually associated with deep sleep and are increased during aging or accelerated cognitive deterioration) and a significant increase in beta waves (normal waking consciousness). Cognition enhancing effect was probably correlated with alpha-GPC's stimulatory effect on brain acetylcholine (a neurotransmitter that increases the release of growth hormone) release. Acetylcholine has been shown to increase at the onset of the standard sustained attention task.

Well controlled clinical trials are reporting that glycerophosphocholine improves attention and word recalling in young, improves cognition, mood and behaviour in people with memory decline, aids in recovery from brain trauma, can be used to boost growth hormone, and can partially revitalize aging brain. Alpha-GPC also supports other transmitter systems such as dopamine, norepinephrine, and gamma-aminobutyric acid (GABA) (in vitro and in vivo evidence).

Siberian Ginseng, eleuthero, a small, woody shrub native to the mountain forests of Northeastern Asia. It has been used for centuries in China and Russia to prevent colds and flu, increase energy, vitality, and longevity. It has been shown in both in vitro and animal models to increase endurance, muscle strength, increase memory and learning, anti-fatigue, increase immunity, and anti-depressant effects.

The preferred amount of Siberian Ginseng in the present gum with 400-2500 mg of Gum base is 30-200 mg.

The adaptogenic properties of siberian ginseng are believed to be due to its effects on hypothalamic-pituitary-adrenal axis, resulting in elevated plasma corticotropin and corticosteroids levels. A thorough review of the human studies to withstand adverse conditions (heat, noise, motion, work-load increase and exercise) to improve auditory disturbances, mental alertness, intellectual work capacity, work output, quality of work and athletic performance concluded the evidence is extensive.

For both ashwagandha and Rhodiola, the root is used to make medicine. Both herbs are employed by herbalists to combat stress and fatigue, support the nervous system, enhance endurance and address infertility. Within the realm of scientific study, Ashwagandha's strongest therapeutic capabilities lie in reducing pain and swelling in arthritis and supporting immune system function. Rhodiola's prominent scientifically supported functions are enhancing the body's resistance to stress, increasing physical endurance, and diminishing fatigue and anxiety. In addition to their main medicinal uses, Ashwagandha and Rhodiola each have unique therapeutic nuances and therefore taken together are incredibly “synergistic” and give you calm, focused, and very controlled energy throughout the day.

Examples of other substances that have a great effect on cognitive enhancements and fast learning abilities are listed below.

Phosphatidylserine can slow and even reverse the decline of learning, mood, memory, concentration, word recall related to dementia or age-related cognitive impairment in middle-aged and elderly subjects. Phosphatidylserine is a natural nutrient that has been found to block the harmful effects of cortisol. Phosphatidylserine is not a neurotransmitter but a special form of phospholipid which supports many neurotransmitters improving cognitive function, clearing depression, and enhancing memory and mood, phosphatidylserine (PS) blunts cortisol and helps us deal with stress. Phosphatidylserine is also vital to cellular communication and the orchestration of so many important tasks. These include stimulating the release of many brain neurotransmitters, regulating the availability of glucose and activating the transport of nutrients into the cells acting as a type of gate-keeper. For our brain to learn efficiently, it needs adequate levels of phosphatidylserine. FDA-qualified health claim states that phosphatidylserine may reduce the risk of dementia and cognitive dysfunction.

Phosphatidylserine is thus absolutely vital. Aging slows the production of phosphatidylserine to sub-optimal levels that preclude us from functioning at full mental capacity.

For memory formation and learning, one of the most important brain chemicals is called acetylcholine. This neurotransmitter is derived from a precursor called choline and alpha GPC is among the top sources of this essential nutrient. Acetylcholine is utilized by the hippocampus to create memories. Alpha GPC repairs and maintains the cells so that the neurons communicate better and faster. Alpha GPC thus improves mood, and boosts mental energy. The extra choline can increase alertness and clarity of thought. Alpha GPC is prized for its ability to improve memory. Its high bioavailability makes it a great source of choline for producing the neurotransmitter acetylcholine. Alpha GPC boosts the development of new brain cells. And enhances your brain's ability to repair damaged cell membranes.

L-Tyrosine,—is one of the most important amino acids, which is used in the synthesis of structural proteins. Tyrosine is excellent for the brain as it is a neurotransmitter and activates adrenaline, dopamine and serotonin in the brain and enhance the ratio of norepinephrine that enhance alertness and awareness as well as increase of the production of epiphrine that reduces blood supply during stress. Other hypothesized that tyrosine may improve physical performance only if the exercise being done produces enough cognitive stress and depletes dopamine or norepinephrine levels, which is assumable in pressured conditions such as competitions.

Lion's Mane mushroom has nerve regenerating capability and enhances nerve growth, both in the brain and throughout the body. Lion's Mane extracts decreased wakefulness at the end of the active phase in mice. Furthermore, some components of Lion's Mane can advance the sleep-wake cycle. Therefore, it has been suggested that Lion's Mane may help in conditions with circadian clock impairments, such as dementia, Alzheimer's and delayed sleep phase disorder.

Reishi—The reishi mushroom (Ganoderma lucidum), an edible type of medicinal fungus that has been used for various healing abilities for thousands of years, is a true “superfood.” Also known as Ling Zhi in Chinese, these mushrooms are strongly anti-inflammatory and tied to longevity, better immune function and mental clarity—perhaps that's why they've adopted the nickname “king of mushrooms.”

As described in the book “Herbal Medicine: Biomolecular and Clinical Aspects,” in Chinese the name for the reishi mushroom represents “a combination of spiritual potency and essence of immortality . . . it's tied to success, well-being, divine power, and longevity. Among cultivated mushrooms, G. lucidum is unique in that its pharmaceutical rather than nutritional value is paramount.”

Throughout history in holistic medicine practices, including Traditional Chinese Medicine (TCM), reishi mushrooms have been considered to be adaptogen herb-like substances, meaning they help us deal with the negative effects of stress—such as increased inflammation, depleted energy levels, damaged blood vessels and various types of hormonal imbalances. Studies have repeatedly shown that reishi mushrooms have antioxidant abilities that allow them to strengthen the body's defenses against cancer, autoimmune diseases, heart disease, allergies, infections and more.

Oat-extract—its ability to “initiate the sleep” and not just gives you a longer sleep time (L-Theanine) but plays a huge role of the Melatonin and L-Tryptophan (affects the serotonin), compounds that are very crucial for an intact “Circadian Rytm”. Deviating from your body's circadian rhythm can lead to neurological and physical problems

Cat's Claw boosts Tryptophan levels which increases serotonin and can provide a calming effect.

Piperine (blackpepper extract) (also known as Bioperine™) play a crucial role due to its ability to increase the bioavailability in other of nutritional compounds. The metabolic process that generates energy at the cellular level in the human body is called thermogenesis. Though thermogenesis has been identified as a key factor in maintaining weight loss, it has also been identified as playing an integral role in utilizing the daily food and nutrients that the human body consumes.

The preferred amount of Piperine in the present gum with 400-2500 mg of Gum base is 2-30 mg.

### Synergistic Effect of the Phytonutrients and Adaptogenic Herbs

Researchers have shown that a nutrient-rich biomass called shilajit can boost CoQ10 efficiency. Shilajit is prized for its ability to carry energy and nutrition into the body. When CoQ10 and shilajit are combined, scientists have discovered improved mitochondrial function, enhancement of the mitochondria's ability to convert food into energy, and increased energy available to tissues. CoQ10 offers a powerful way to help slow, or even reverse, a natural aging process by restoring youthful mitochondrial protection against free radicals. CoQ10 can quickly become depleted in the body because it donates its own electrons in order to neutralize the flood of free radicals. That makes ensuring the body's continued supply of CoQ10 critical and shilajit has been found to stabilize, revitalize, and preserve CoQ10 in its active (ubiquinol) form, boosting the levels of CoQ10 that are critical for protection against mitochondrial aging. The key discovery was that the combination of these two nutrients had a synergistic effect, producing greater ATP recovery than seen with either nutrient alone. In fact, in the brain and blood, the shilajit/CoQ10 combination brought ATP levels back nearly to those of un-exercised control animals! As an added benefit, supplementation with shilajit prevented significant drops in CoQ10 levels in the blood. Ultimately, the synergistic effects of shilajit plus CoQ10 offer an unparalleled option to protect mitochondrial DNA and combat aging.

### Bioavailability of the Phytonutrients and Adaptogenic Herbs

Oral absorption of drug is very important issue especially when the drug is poorly bioavailable, given for long periods and expensive. Poorly bioavailable drugs remain sub-therapeutic because a major portion of a dose never reaches the plasma or exerts its pharmacological effect unless and until very large doses are given which may also lead to serious side effects. Any significant improvement in bioavailability will result in lowering the dose or the dose frequency of that particular drug. Bioenhancers or bioavailability enhancers are mostly the plant based molecules which promote the biological activity or bioavailability or the uptake of drugs in combination therapy. The nature of a drug formulation can directly influence its dissolution, as well as its rate and extent of absorption after oral administration. Chewing gum formulations have been evaluated for several drugs including aspirin, verapamil and most recently nicotine. The gum formulations offer several advantages over the tablet or liquid formulations, such as: most of the drug released from the gum through mastication is believed to be absorbed via the buccal cavity. Absorption through the buccal cavity is considered faster due to its extensive vascularization, especially for low molecular weight hydrophobic agents. As the onset of drug action in most instances is dependent on speed of delivery, a faster absorption results in a shorter duration for a dynamic response. Drugs absorbed via the buccal cavity bypass intestinal and hepatic first pass metabolism, which potentially increases their extent of absorption.

In the present invention the problem of bioavailability of the active ingredients such as Shilajit, Ashwagandha, Coenzyme Q10 and Siberian Ginseng is solved by providing a functional gum formulation comprising piperine. With the help of piperine and absorption through the buccal cavity provided by the gum, the effect of the active ingredients is surprisingly quick and potent and also long-lasting as the gum provides slow-release properties.

In an embodiment, active ingredients of a chewing gum boosting in mental clarity and concentration are selected from the group consisting of:

Shilajit, L-Tyrosine, L-Theanine, Vitamin B6, Vitamin B12, Catsclaw root extract (Uncaria tomentosa), L-Alpha glycerylphosphorylcholine (alpha-GPC), Bacopa monnieri, Ashwagandha (Withania somnifera), Lion's Mane, L-Citrullin, Vinpocetine, piperine (blackpepper extract), Folic acid, Oat extract (Avena sativa), Resveratrol, Pterostilbene, Phosphatidylserine (preferably GMO free), and Rhodiola.

In preferred embodiments, active ingredients of a chewing gum boosting in mental clarity and concentration are selected from the following groups:
Shilajit, Ashwagandha, Coenzyme Q10, Phosphatidylserine (preferably GMO free), L-Alpha glycerylphosphorylcholine (alpha-GPC) and piperine (blackpepper extract); or
Shilajit, Ashwagandha, Coenzyme Q10, Phosphatidylserine (preferably GMO free), Bacopa monnieri and piperine (blackpepper extract).

In an embodiment, active ingredients of a chewing gum providing alleviation of fatigue and enhancement of energy are selected from the group consisting of:

Shilajit, Coenzyme Q10, Phosphatidylserine, Glycerylphosphorycholine (Alpha GPC), Siberian ginseng, Ganoderma lucidum (Reishi), Hericium erinaceus (Lion's mane), Vitamin C, Vitamin B6, piperine (blackpepper extract),

In preferred embodiments, active ingredients of a chewing gum providing alleviation of fatigue and enhancement of energy are selected from the following groups:
Shilajit, Coenzyme Q10, Phosphatidylserine, Glycerylphosphorycholine (Alpha GPC), Siberian ginseng, Ganoderma lucidum (Reishi), Hericium erinaceus (Lion's mane) and piperine (blackpepper extract); or
Shilajit, Coenzyme Q10, Phosphatidylserine, Glycerylphosphorycholine (Alpha GPC), Siberian ginseng, and piperine (blackpepper extract)

In another embodiment, active ingredients of a chewing gum boosting in mental clarity and concentration are selected from the group consisting of: L-Tyrosine, L-Theanine, Oat extract (Avena sativa), Phosphatidylserine, vitamin B6, Catsclaw root extract (Uncaria tomentosa), L-Alpha glycerylphosphorylcholine (alpha-GPC), Guayusa, Bacopa monnieri, Ashwagandha (Withania somnifera), Lion's Mane, L-Citrullin, Vinpocetine, piperine (blackpepper extract), Pterostilbene, Phosphatidylserine (preferably GMO free), and Rhodiola.

In preferred embodiments, active ingredients of a chewing gum boosting in mental clarity and concentration selected from the following groups:
Ashwagandha, Rhodiola, Guayusa, Phosphatidylserine (preferably GMO free), and piperine (blackpepper extract); or
Ashwagandha, Rhodiola, L-Theanine, piperine (blackpepper extract), and L-Alpha glycerylphosphorylcholine (alpha-GPC).

In another embodiment, active ingredients of a chewing gum providing alleviation of fatigue and enhancement of energy are selected from the group consisting of: citrulline malate, L-Tyrosine, alpha-glycerylphosphorycholine, Quercus robur wood extract “QR”, coconut oil powder, shilajit, guayusa, Vinpocetine, vitamin B6, piperine (blackpepper extract), CoQ10, Lion's mane, and Reishi.

In preferred embodiments, active ingredients of a chewing gum providing alleviation of fatigue and enhancement of energy are selected from the following groups:
citrulline malate, L-Tyrosine, Coconut Oil powder, Shilajit, Guayusa, and piperine (blackpepper extract); or
citrulline malate, alpha-glycerylphosphorycholine, Coconut Oil powder, Shilajit,

Guayusa, piperine (blackpepper extract)

In a preferred embodiment, the functional chewing gum of the present invention may also contain further active ingredients boosting in mental clarity and concentration and/or providing alleviation of fatigue and enhancement of energy.

A preferred amount of gum base in the final chewing gum is 30-75% by weight of the chewing gum before any optionally applied coating, such as 35-70% by weight of the chewing gum or 40-65% by weight of the chewing gum or 45-60% by weight of the chewing gum.

The formulation of gum bases can vary depending on the particular product to be prepared.

The gum base may optionally contain synthetic elastomers such as polyvinyl acetate or vinyl laurate-vinyl acetate or a copolymer thereof.

Other synthetic elastomers may be selected from the group consisting of styrene-butadiene copolymers, polyisobutylene, isobutylene-isoprene copolymers, polyurethane and polyethylene.

The gum base may further comprise: 0 to 40% by weight waxes, 5 to 35% by weight softeners other than waxes, such as plasticizers, fats and emulsifiers, 0 to 50% by weight filler, and 0 to 5% by weight of miscellaneous ingredients such as antioxidants, colorants, etc. In an embodiment of the invention, the functional chewing gum comprises further chewing gum ingredients selected from the group consisting of flavors, dry-binders, tableting aids, anti-caking agents, emulsifiers, antioxidants, enhancers, absorption enhancers, high intensity sweeteners, softeners, colors, active ingredients, water-soluble indigestible polysaccharides, water-insoluble polysaccharides or any combination thereof.

In an embodiment of the invention the chewing gum comprises flavor. Flavor may typically be present in amounts between 0.01 and 10% by weight of the chewing gum, such as between 0.01 and 5% by weight of the chewing gum.

Non-exhaustive examples of flavors suitable in embodiments of the present invention are coconut, coffee, chocolate, vanilla, grape fruit, orange, lime, menthol, liquorice, caramel aroma, honey aroma, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, anise, cedar leaf oil, and oils of the fruits mentioned above.

Petroleum waxes aid in the curing of the finished gum made from the gum base as well as improve shelf life and texture. Wax crystal size influences the release of flavor. Waxes and fats are also conventionally used for the adjustment of the texture and for softening of the chewing gum base when preparing chewing gum bases.

Antioxidants prolong shelf life and storage of a gum base, finished gum or their respective components including fats and flavor oils. Antioxidants suitable for use in gum base include butylated hydroxyanisole, butylated hydroxytoluene, betacarotenes, tocopherols, acidulants such as Vitamin C, propyl gallate, other synthetic and natural types or mixtures thereof.

Further chewing gum ingredients, which may be included in the functional chewing gum according to the present invention, include surfactants and/or solubilizers.

In some embodiments, one or more colors can be included in the chewing gum.

High intensity artificial sweetening agents can also be used according to preferred embodiments of the invention. Preferred high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside and the like, alone or in combination.

In order to provide longer lasting sweetness and flavor perception, it may be desirable to encapsulate or otherwise control the release of at least a portion of the artificial sweeteners.

Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another chewing gum component such as a resinous compound.

Combinations of sugar and/or non-sugar sweeteners may be used in the chewing gum.

A chewing gum and/or gum base may, if desired, include one or more fillers/texturizers including as examples, magnesium- and calcium carbonate, sodium sulphate, ground limestone, silicate compounds such as magnesium- and aluminum silicate, kaolin and clay, aluminum oxide, silicium oxide, talc, titanium oxide, mono-, di- and tri-calcium phosphates, cellulose polymers, such as wood, and combinations thereof.

The present invention is further described in the following Experimental Section, which is not intended to limit the scope of the invention.

### EXAMPLES

### Example 1

### Ingredients of a Chewing Gum Boosting in Mental Clarity and Concentration

400-1000 mg Gum base

### 500 mg Ashwagandha

### 300 mg Rhodiola

### 200 mg Guayusa

300 mg Phosphatidylserine GMO free
10 mg Bioperine (Piperine—Blackpepper extract)

The mg levels of the active ingredients are the daily dosage which equals to 4 gums a day. For instance, 500 mg of Ashwagandha equals 125 mg/gum servings etc.

### Example 2

### Ingredients of a Chewing Gum Boosting in Mental Clarity and Concentration

400-1000 mg Gum base

### 500 mg Ashwagandha

### 300 mg Rhodiola

### 200 mg L-Theanine

10 mg Bioperine (Piperine—Blackpepper extract)
300 mg L-Alpha glycerylphosphorylcholine (GPC)

The mg levels of the active ingredients are the daily dosage which equals to 4 gums a day. For instance, 500 mg of Ashwagandha equals 125 mg/gum servings etc.

### Example 3

### XrZice—Alleviation of Fatigue and Enhancement of Energy

400-1000 mg Gum base

### 500 mg Citrulline Malate

### 500 mg L-Tyrosine

450 mg (30 ml) Coconut Oil powder

### 650 mg Shilajit

### 400 mg Guayusa

### 10 mg Bioperine

The mg levels of the active ingredients are the daily dosage which equals to 4 gums a day. For instance, 500 mg of L-Tyrosine equals 125 mg/gum servings etc.

### Example 4

### XrZice 1—Alleviation of Fatigue and Enhancement of Energy

400-1000 mg Gum base

### 500 mg Citrulline Malate

### 300 mg Alpha Glycerylphosphorycholine

450 mg (30 ml) Coconut Oil powder

### 650 mg Shilajit

### 400 mg Guayusa

### 10 mg Bioperine

The mg levels of the active ingredients are the daily dosage which equals to 4 gums a day. For instance, 500 mg of Citrulline Malate equals 125 mg/gum servings etc.

### Example 5

### Ingredients of a Chewing Gum Boosting in Mental Clarity and Concentration

400-1000 mg Gum base

### 300 mg L-Tyrosine

### 200 mg L-Theanine

150 mg Oat extract (Avena sativa)

### 300 mg Phosphatidylserine

### 10 mg Vitamin B6

350 mg Catsclaw root extract (Uncaria tomentosa)
300 mg (L-Alpha glycerylphosphorylcholine (GPC)
350 mg Bacopa monnieri
500 mg Ashwagandha (Withania somnifera)

### 250 mg Lion's Mane

### 300 mg L-Citrullin

### 10 mg Vinpocetine

10 mg Bioperine (Blackpepper extract)

### 100 mg Pterostilbene

### 300 mg Rhodiola

The mg levels are the daily dosage which equals to 4 gums a day. For instance, 500 mg of Ashwagandha equals 125 mg/gum servings etc.

### Example 6

### Ingredients of a Chewing Gum Providing Alleviation of Fatigue and Enhancement of Energy

400-1000 mg Gum base

### 300 mg Alpha Glycerylphosphorycholine

500 mg Quercus robur wood extract “QR”
450 mg (30 ml) Coconut Oil powder

### 650 mg Shilajit

### 500 mg Guayusa

10 mg bioperine

### 10 mg Vinpocetine

### 10 mg Vitamin B6

### 300 mg CoQ10

### 400 mg Lionsmane

### 400 mg Reishi

The mg levels are the daily dosage which equals to 4 gums a day. For instance, 500 mg of “QR” equals 125 mg/gum servings etc.

### Example 7

### Ingredients of a Chewing Gum Boosting in Mental Clarity and Concentration

400-2500 mg Gum base

### 75 mg Shilajit

### 62.5 mg L-Tirosine

43.75 Bacopa monnieri
31.25 mg Phosphatidylserine GMO free

### 31.25 mg Glycerophosphocholine

### 31.25 mg Ashwagandha

### 18.75 mg Coenzyme Q10

2-5 mg Bioperine (Piperine—Blackpepper extract)

The mg levels of the active ingredients are the daily dosage which equals to 4 gums a day. For instance, 300 mg of Shilajit equals 75 mg/gum servings etc.

### Example 8

### Ingredients of a Chewing Gum Boosting in Mental Clarity and Concentration

400-2500 mg Gum base

### 75 mg Shilajit

### 62.5 mg L-Tirosine

### 31.25 mg Ashwagandha

31.25 mg L-Alpha glycerylphosphorylcholine (GPC)

### 18.75 mg Coenzyme Q10

### 12 mg Resveratrol

### 2 mg Vitamin B6

2-5 mg Bioperine (Piperine—Blackpepper extract)

The mg levels of the active ingredients are the daily dosage which equals to 4 gums a day. For instance, 300 mg of Shilajit equals 75 mg/gum servings etc.

### Example 9

### XrZice2—Alleviation of Fatigue and Enhancement of Energy

400-2500 mg Gum base

### 75 mg Shilajit

37.5 mg Phosphatidylserine GMO free

### 37.5 mg Glycerophosphocholine

### 37.5 mg Siberian Ginseng

### 18.75 mg Coenzyme Q10

### 2 mg Vitamin B6

2-5 mg Bioperine (Piperine—Blackpepper extract)

The mg levels of the active ingredients are the daily dosage which equals to 4 gums a day. For instance, 75 mg of Shilajit equals 300 mg/gum servings etc.

### Example 10

### XrZice3—Alleviation of Fatigue and Enhancement of Energy

400-2500 mg Gum base

### 75 mg Shilajit

37.5 mg Phosphatidylserine GMO free

### 37.5 mg Siberian Ginseng

37.5 Ganoderma lucidum
37.5 Hericium erinaceus

### 18.75 mg Coenzyme Q10

### 12 mg Resveratrol

### 2 mg Vitamin B6

2-5 mg Bioperine (Piperine—Blackpepper extract)

The mg levels of the active ingredients are the daily dosage which equals to 4 gums a day. For instance, 300 mg of Sjhilajit equals 75 mg/gum servings etc.

### Example 11

### Ingredients of a Chewing Gum Boosting in Mental Clarity and Concentration

400-2500 mg Gum base

### 62.5 mg L-Tyrosine

43.75 mg Bacopa monnieri
31.25 mg Ashwagandha (Withania somnifera)

### 31.25 mg Phosphatidylserine

31.25 mg Catsclaw root extract (Uncaria tomentosa)
31.25 mg (L-Alpha glycerylphosphorylcholine (GPC)

### 2 mg Vitamin B6

2-5 mg Bioperine (Piperine—Blackpepper extract)

The mg levels are the daily dosage which equals to 4 gums a day. For instance, 125 mg of Ashwagandha equals 31.25 mg/gum servings etc.

### Example 12

### Ingredients of a Chewing Gum Providing Alleviation of Fatigue and Enhancement of Energy

400-1000 mg Gum base

### 250 mg Shilajit

### 100 mg CoQ10

### 37.5 mg Alpha Glycerylphosphorycholine

37.5 mg Siberian ginseng
2-5 mg Bioperine (Piperine—Blackpepper extract)

The mg levels are the daily dosage which equals to 4 gums a day. For instance, 250 mg of Shilaji equals 62.5 mg/gum servings etc.

1-14. (canceled)

15. A chewing gum comprising a gum base and active ingredients effective in boosting mental clarity and concentration, wherein said active ingredients comprise at least Shilajit, Ashwagandha, Phosphatidylserine, L-Alpha glycerylphosphorylcholine (alpha-GPC), and piperine (blackpepper 5 extract).

16. The chewing gum according to claim 15, further comprising an additional active ingredient selected from the group consisting of Bacopa monnieri, L-Tyrosine, vitamin B6, Folic acid, Catsclaw root extract (Uncaria tomentosa), L-Citrullin, Resveratrol, Folic acid, L-Theanine, Vinpocetine, Vitamin C, Vitamin B1, Vitamin B5, Vitamin B6, and Pterostilbene.

17. The chewing gum according to claim 16, wherein the additional active ingredient comprises Bacopa monnieri.

18. A chewing gum comprising a gum base and active ingredients effective in providing alleviation of fatigue and enhancement of energy, wherein said active ingredients comprise at least Shilajit, Coenzyme Q10, Phosphatidylserine, Siberian Ginseng, and piperine (blackpepper extract).

19. The chewing gum according to claim 18, further comprising an additional active ingredient selected from the group consisting of Ganoderma lucidum (Reishi), Glycerophosphocholine, Hericum erinaceus (Lion's mane), Zinc, Iodine, Magnesium, Vitamin B1, Vitamin B2, Vitamin B5, Vitamin B6, and Vitamin B12.

20. The chewing gum according to claim 19, wherein said additional active ingredient comprises Glycerophosphocholine.

21. A chewing gum comprising a gum base and active ingredients effective in boosting in mental clarity and concentration, wherein said active ingredients comprise at least Ashwagandha, Rhodiola, Guayusa, Phosphatidylserine, and piperine.

22. The chewing gum according to claim 21, further comprising an additional active ingredient selected from the group consisting of: L-Tyrosine, L-Theanine, Oat extract (Avena sativa), vitamin B6, Catsclaw root extract (Uncaria tomentosa), L-Alpha glycerylphosphorylcholine (alpha-GPC), Bacopa monnieri, Lion's Mane, L-Citrullin, Vinpocetine, and Pterostilbene.

23. A chewing gum comprising a gum base and active ingredients effective in boosting in mental clarity and concentration, wherein said active ingredients comprise at least Ashwagandha, Rhodiola, L-Theanine, L-Alpha glycerylphosphorylcholine (alpha-GPC), and piperine (blackpepper extract).

24. The chewing gum according to claim 23, further comprising an active ingredient selected from the group consisting of: L-Tyrosine, Oat extract (Avena sativa), Phosphatidylserine, vitamin B6, Catsclaw root extract (Uncaria tomentosa), Guayusa, Bacopa monnieri, Lion's Mane, L-Citrullin, Vinpocetine, Pterostilbene, and Phosphatidylserine.

25. A chewing gum comprising a gum base and active ingredients effective in providing alleviation of fatigue and enhancement of energy, wherein said active ingredients comprise at least citrulline malate, L-Tyrosine, Coconut Oil powder, Shilajit, Guayusa, and piperine (blackpepper extract).

26. The chewing gum according to claim 25, further comprising an additional active ingredient selected from the group consisting of: alpha-glycerylphosphorycholine, Quercus robur wood extract, Vinpocetine, vitamin B6, CoQ10, Lion's mane, and Reishi.

27. A chewing gum comprising a gum base and active ingredients effective in providing alleviation of fatigue and enhancement of energy, wherein said active ingredients comprise at least citrulline malate, alpha-glycerylphosphorycholine, Coconut Oil powder, Shilajit, Guayusa, and piperine (blackpepper extract).

28. The chewing gum according to claim 27, further comprising an active ingredient selected from the group consisting of: L-Tyrosine, Quercus robur wood extract, Vinpocetine, vitamin B6, CoQ10, Lion's mane, and Reishi.
